(19) United States
(12) Patent Application Publication
(10) Pub. No.: US 2008/0118918 A1
(43) Pub. Date: May 22, 2008

(54) Compositions and Methods For Determining and Predicting Treatment Responses For Depression and Anxiety

(75) Inventors: Julio Licinio, Miami, FL (US); Ma-Li Wong, Miami, FL (US); Kristopher J.L. Irizarry, Sugarloaf, CA (US); Katherine Misouras Irizarry, Sugarloaf, CA (US)

Correspondence Address: MORRISON & FOERSTER LLP, 12531 HIGH BLUFF DRIVE, SUITE 100, SAN DIEGO, CA, 92130-2040, US

(73) Assignee: THE REGENTS of the University of California, Oakland, CA (US)

(21) Appl. No.: 11/573,689

(22) PCT Filed: Aug. 12, 2005
(86) PCT No.: PCT/US05/28790

§ 371 Date: Dec. 10, 2007

Related U.S. Application Data
(60) Provisional application No. 60/601,361, filed on Aug. 13, 2004.

(51) Int. Cl. C12Q 1/68; G01N 33/574

(52) U.S. Cl. 435/6; 435/7.21

(57) ABSTRACT

The invention provides genetic means, including compositions (e.g., kits) and methods, to predict the efficacy of a treatment in depression and anxiety and to predict a patient's response to a particular a specific medication or treatment. The invention provides methods for diagnosing the presence of a psychiatric disorder or determining the outcome of a treatment for a psychiatric disorder. The invention provides methods for methods for diagnosing the presence of a psychiatric disorder in an individual by determining what corticotropin-releasing hormone receptor 1 (CRHR1) protein or transcript isoforms are expressed in an individual. The invention provides methods for diagnosing the presence of a psychiatric disorder in an individual by determining what protein or transcript isoforms are expressed in an individual wherein the protein is involved in the hypothalamic-pituitary-adrenal (HPA) axis pathway.

### CROSS-REFERENCES TO RELATED APPLICATIONS

This application claims benefit of priority under 35 U.S.C. § 119(e) from provisional application U.S. Ser. No. 60/601,361 filed 13 Aug. 2004, the contents of which are incorporated herein by reference and for all purposes.

### FEDERAL FUNDING

This invention was produced in part using funds from the Federal government under NIH Grant Nos. NIH grants GM61394, HL65899, RR017365, MH062777, RR000865, K30HL04526, RR16996, HG002500, DK063240 and T32 MH017140. Accordingly, the Federal government has certain rights in this invention.

### TECHNICAL FIELD

This invention relates to medicine, psychiatry and molecular cellular. In one aspect, the invention provides genetic means, including compositions and methods, to predict the efficacy of a treatment in depression in a subpopulation of patients and to predict a patient's response to a particular a specific medication or treatment. In one aspect, the invention provides methods for diagnosing the presence of a psychiatric disorder or determining the outcome of a treatment for a psychiatric disorder. In one aspect, the invention provides methods for diagnosing the presence of a psychiatric disorder in an individual by determining what corticotropin-releasing hormone receptor 1 (CRHR1) protein or transcript isoforms are expressed in an individual.

### BACKGROUND

Major depression is a common and complex disorder of gene-environment interactions. The specific genetic substrates and precipitating environmental factors have not yet been elucidated. The disorder affects 10% of males and 20% of females and has a point prevalence of 3%. Its cost to the U.S. economy exceeds 100 billion dollars per year. Over twenty drugs are approved by the U.S. Food and Drug Administration for treatment of depression, each one with efficacy of approximately 60%. Various subgroups of patients respond differently to each drug, so that if multiple trials are conducted, eventually 85% of patients will respond. Because there are no clinical or biomarker predictors of treatment response, the assignment of a depressed patient to a drug is based solely on chance or on attempts to minimize side effects that are more likely to occur with a specific medication.

Antidepressants of various classes have been shown to suppress corticotropin-releasing hormone (CRH) receptor (CRR gene expression) in rodents as well as in depressed and healthy humans. It has been shown that there is an association between the disruption of the hypothalamic-pituitary-adrenal (HPA) axis function and depression, including, for example, increased 24-h elevations in cortisol production, lack of suppression of plasma cortisol levels by dexamethasone, increased concentrations of CRH in cerebrospinal fluid (CSF), dysregulation of HPA responses to exogenous CRH administration and loss of the negative correlation between plasma cortisol and continuously-collected CSF CRH. It has been proposed that suppression of CRH activity is a common, final effect of antidepressant treatment. It also has been demonstrated that variants in the corticotropin-releasing hormone receptor type 1 (CRHR1) gene can be associated with the response to inhaled steroids in asthma.

Drugs acting through multiple mechanisms of action have been shown to have equal effects on downregulating CNS CRH gene expression. Based on extensively replicated work from several groups, it has therefore been proposed that suppression of CRH activity is a common, final effect of antidepressant treatment with, for example, tricyclic drugs, such as imipramine (or its metabolite desipramine), selective serotonin reuptake inhibitors, such as fluoxetine, and also with non-pharmacologic treatment such as electroconvulsion. Because of the consensus on the role of CRHR1 in depression, CRHR1 antagonists have been developed and successfully used in clinical research contexts as antidepressants, without long-term effects on HPA responses to stress.

A genetic association of a phenotype to an antidepressant treatment response has been studied by several groups. The most consistent findings have been of associations of antidepressant treatment responses with the insert/delete polymorphism of the upstream regulatory region of the serotonin transporter gene. Other genes shown to be associated with antidepressant treatment response include tryptophan hydroxylase and the serotonin 2_{A} receptor.

### SUMMARY

The invention provides compositions and methods for associating specific corticotropin-releasing hormone receptor type 1 (CRHR1) sequence variations, so-called haplotypes, with different antidepressant-mediated responses in a human subpopulation, particularly, a subpopulation having a diagnosis of major depression and having high levels of anxiety. Thus, the compositions and methods of the invention can be used to predict a sub-population of patients (as defined by a haplotypes) response to antidepressant and to aid in monitoring, evaluating and adjusting an on-going regimen of antidepressant drug therapy. In particular, the compositions and methods of the invention can be used to predict and monitor treatment responses in a phenotypic subgroup of depressed patients, i.e., a high-anxiety depressed patient subpopulation, by determining the presence, or absence, of homozygozity for the GAG haplotype of the corticotropin-releasing hormone receptor type 1 (CRHR1) gene (or “haplotype 1”, as discussed in Example 1, below). Significant association of antidepressant treatment response and a genotype that is homozygous for a CRHR1, or “GAG,” haplotype (presence of haplotype-tag single nucleotide polymorphisms (htSNPs) identified as rs1876828 (SEQ ID NO:2), rs242939 (SEQ ID NO:6) and rs242941 (SEQ ID NO:7) occurred only in the high anxiety, depressed group.

While the invention is not limited to practicing the compositions and methods on any particular individual or subpopulation, because the “CRH-R1” or “GAG” haplotype described herein is relatively frequent in American Latino subpopulations, the compositions and methods of the invention can be particularly useful for predicting antidepressant treatment response in Mexican Americans.

This invention first demonstrates that there is stratification of the response to antidepressant treatment in high-anxiety depressed patients according to a haplotype of CRHR1. As discussed in detail in Example 1, below, utilizing the htSNPs rs1876828 (SEQ ID NO:2), rs242939 (SEQ ID NO:6) and rs242941 (SEQ ID NO:7), it was shown that homozygozity for the GAG haplotype was associated with a 70% greater reduction in HAM-A scores (63.1±4.5% in homozygous and 37.1±6.9% in heterozygous, P=0.002) and 31% greater reduction in HAM-D scores after treatment (67.3±4.3% in homozygous and 51.2±6.0% in heterozygous, P=0.03).

The invention provides methods for determining a subject's responsiveness to a psychiatric disorder therapy, wherein the subject is diagnosed with depression and anxiety, the method comprising the steps of: (a) providing a nucleic acid-comprising sample from the subject; (b) analyzing the sample and detecting whether the subject is homozygous for a CRHR1, or “GAG,” haplotype (homozygous for the haplotype-tag single nucleotide polymorphisms (htSNPs) rs1876828 (SEQ ID NO:2), rs242939 (SEQ ID NO:6) and rs242941 (SEQ ID NO:7), wherein the presence of homozygosity for the CRHR1 genotype correlates with responsiveness to the therapy. The invention also provides methods screening a subject to determine or predict the subject's responsiveness to a psychiatric disorder therapy comprising use of an agent which prevents or treats a psychiatric disorder associated with the corticotropin-releasing hormone receptor 1 (CRHR1) gene, comprising: (a) providing a nucleic acid-comprising sample from the subject; (b) detecting whether the subject is homozygous for a CRHR1, or “GAG,” haplotype (homozygous for the haplotype-tag single nucleotide polymorphisms (htSNPs) rs1876828 (SEQ ID NO:2), rs242939 (SEQ ID NO:6) and rs242941 (SEQ ID NO:7), wherein the presence of homozygosity for the CRHR1 genotype correlates with responsiveness to the therapy. Thus, the methods of the invention can be used by a health professional to develop a treatment therapy or regimen, or help evaluate or critique an on-going treatment regimen.

In one aspect of the methods, wherein the subject is diagnosed (e.g., as diagnosed according to the Structured Clinical Interview for DSM-IV, as discussed below, or any other accepted diagnostic protocol) as having a psychiatric disorder comprising depression and anxiety. In one aspect, the psychiatric disorder comprises major depression and anxiety disorder. In one aspect, the psychiatric disorder therapy comprises treatment with an antidepressant agent, e.g., tricyclic antidepressants, selective serotonin reuptake inhibitors and/or CRHR1 antagonists or equivalent drugs.

In one aspect of the methods, the nucleic acid-comprising sample comprises a blood sample, a saliva sample or a cell sample. The cell sample can comprise a cell from any source, e.g., a biopsy, a buccal or a skin scraping or sample.

In one aspect of the methods, the genotype of the subject with respect to the CRHR1, or “GAG,” haplotypes, is determined by amplification genotyping, in situ hybridization techniques, DNA array (biochip) analysis and/or direct DNA sequencing. The amplification genotyping can comprise a polymerase chain reaction (PCR).

In one aspect of the methods, the subject is selected from a subpopulation of patients, e.g., a subpopulation of patients comprising a Mexican-American subpopulation.

The invention provides kits suitable for determining a subject's responsiveness to a psychiatric disorder therapy, wherein the subject is diagnosed with depression and anxiety, said kit comprising (a) material for determining whether the subject is homozygous for a CRHR1, or “GAG,” haplotype (homozygous for the haplotype-tag single nucleotide polymorphisms (htSNPs) rs1876828 (SEQ ID NO:2), rs242939 (SEQ ID NO:6) and rs242941 (SEQ ID NO:7); (b) suitable packaging material; and optionally (c) instructional material for use of said kit. In one aspect of the kit, the material comprises at least one nucleic acid that specifically binds to the htSNPs rs1876828 (SEQ ID NO:2), rs242939 (SEQ ID NO:6) and rs242941 (SEQ ID NO:7). The nucleic acid that specifically binds to the htSNPs can comprise at least one polymerase chain reaction (PCR) primer or a hybridization probe (e.g., for automated, Southern or equivalent analysis capable of determining genotype and whether the subject is homozygous for a CRHR1). The kit can further comprise material to process a nucleic acid-comprising biological sample.

The invention also provides methods for determining a nucleotide polymorphism associated with the presence of a psychiatric disorder in an individual comprising (a) sequencing all or a portion of the nucleotide sequence of chromosome 17q11-q22 from the individual, comparing the 17q11-q22 sequence from the individual to chromosome 17q11-q22 sequences in individuals having the same psychiatric disorder, and determining the presence or absence of a nucleotide polymorphism in individuals having the same psychiatric disorder; and, (b) correlating the diagnosis of the psychiatric disorder to the presence or absence of the nucleotide polymorphism, wherein optionally the psychiatric disorder therapy is a depression.

The invention also provides methods for diagnosing the presence of a psychiatric disorder in an individual by determining a nucleotide polymorphism in an individual comprising (a) sequencing all or a portion of the nucleotide sequence of chromosome 17q11-q22 from the individual, comparing the 17q11-q22 sequence from the individual to chromosome 17q11-q22 sequences in individuals having the same psychiatric disorder, and determining the presence or absence of a nucleotide polymorphism in individuals having the same psychiatric disorder; and, (b) correlating the presence or absence of the nucleotide polymorphism to the presence or absence of a psychiatric disorder, wherein optionally the psychiatric disorder therapy is a depression.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIG. 1 illustrates the frequencies of CRHR1 haplotypes/subject in a population of depressed and control patients, as described in detail in Example 1, below.

FIGS. 2a and 2b illustrate data showing the percent decrease in HAM-D, FIG. 2(a), and HAM-A, FIG. 2(b), across 8 weeks of double-blind antidepressant treatment in a subpopulation diagnosed with depression and with high levels of anxiety, as described in detail in Example 1, below.

FIGS. 3a and 3b illustrate data showing the percent decrease in HAM-D FIG. 3(a), and HAM-A, FIG. 3(b), across 8 weeks of double-blind antidepressant treatment in a subpopulation diagnosed with depression and with lower levels of anxiety, as described in detail in Example 1, below.

FIG. 4 illustrates data of an eight-week response to daily antidepressant treatment, as described in detail in Example 1, below.

FIG. 5 illustrates data of an eight-week response to daily antidepressant treatment, as described in detail in Example 1, below.

FIG. 6 schematically identifies the htSNPs, identified as rs1876828 (SEQ ID NO:2), rs242939 (SEQ ID NO:6) and rs242941 (SEQ ID NO:7), used to generate the “GAG” haplotype, as described in detail, below.

FIGS. 7, 8 and 9, illustrate alternative CRH-R1 transcript forms generated by alternative splicing events, as described in detail, below.

Like reference symbols in the various drawings indicate like elements.

### DETAILED DESCRIPTION

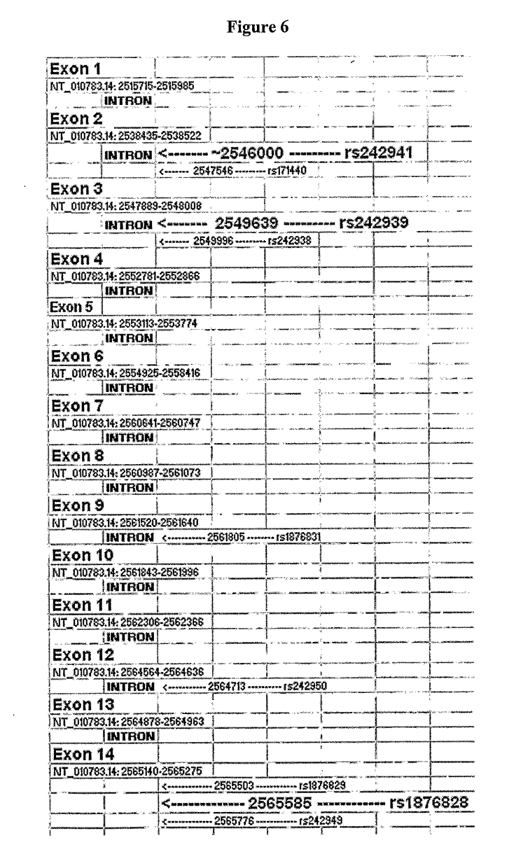

The invention provides compositions and methods for associating specific corticotropin-releasing hormone receptor type 1 (CRHR1) sequence variations, so-called haplotypes, with different antidepressant-mediated responses in a phenotypic subgroup of depressed patients, i.e., a high-anxiety depressed patient subpopulation. In particular, the compositions and methods of the invention can predict an increased response to antidepressants in anxious, depressed patients that are homozygous for the GAG haplotype of CRHR1; identified as the presence of haplotype-tag single nucleotide polymorphisms (htSNPs) identified as rs1876828 (SEQ ID NO:2), rs242939 (SEQ ID NO:6) and rs242941 (SEQ ID NO:7). FIG. 6 schematically illustrates the htSNPs, identified as rs1876828 (SEQ ID NO:2), rs242939 (SEQ ID NO:6) and rs242941 (SEQ ID NO:7), used to generate the “GAG” haplotype used to practice the methods and compositions of the invention.

The present invention provides genetic methods and compositions for the diagnosis, prognosis and treatment of psychiatric disorders associated with the corticotropin-releasing hormone receptor 1 (CRHR1) gene, including depression and/or anxiety disorders and related pathologies. Anxiety disorders encompassed by this invention include panic disorder, obsessive-compulsive disorder, post-traumatic stress disorder, and phobias, including both specific phobias and social phobias. The methods and compositions of the invention utilize polymorphic variations in the CRHR1 gene to screen patients to assess their responsiveness to particular psychiatric therapies, development of diagnostics and therapies for psychiatric disorders associated with the CRHR1 gene, and development of individualized drug treatments based on an individual's genotypic profile with respect to the CRHR1 gene.

In demonstrating the efficacy of the compositions and methods of the invention, as discussed in Example 1, below, the association of CRHR1 genotypes with the phenotype of antidepressant treatment response in 80 depressed Mexican-Americans in Los Angeles who completed a prospective randomized, placebo lead-in, double-blind treatment of fluoxetine or desipramine, with active treatment for eight weeks. Subjects were diagnosed according to the Structured Clinical Interview for DSM-IV (SCID) and included into the study if they had a diagnosis of depression without other confounding medical or psychiatric diagnoses or treatments. All patients were followed weekly and assessed for changes in the Hamilton rating scales for anxiety (HAM-A) and depression (HAM-D). Inclusion criteria in the study included a HAM-D of 18 or higher. Patients were classified into a high anxiety (HA) group if their HAM-A score was 18 or higher and in a low anxiety (LA) group if their HAM-A score was less than 18.

Utilizing the haplotype-tag single nucleotide polymorphisms (htSNPs) rs1876828 (SEQ ID NO:2), rs242939 (SEQ ID NO:6) and rs242941 (SEQ ID NO:7), we tested for haplotypic association between CRHR1 and eight-week response to daily antidepressant treatment. In the HA group (n=54), homozygozity for the GAG haplotype was associated with a relative 70% greater reduction in HAM-A scores (i.e., less anxiety) compared to heterozygous (63.1±4.5% versus 37.1±6.9%, respectively, P=0.002). For HAM-D, GAG haplotype homozygozity was associated with a 31% greater reduction in scores (i.e., less depression) after treatment compared to heterozygous (67.3±4.3% versus 51.2±6.0%, respectively, P=0.03). In those with lower anxiety levels at screening there were no associations between CRHR1 genotype and % change in HAM-A or HAM-D. These data demonstrate an increased response to antidepressants in highly anxious patients homozygous for the GAG haplotype of CRHR1.

In one aspect, the invention is partially based on the observation that subjects having polymorphic variation for the CRHR1 gene demonstrate differential responsiveness to antidepressant drug therapy. In one aspect, the methods of the invention comprise determination of a subject's genotype with respect to the CRHR1 gene. Such genotype, e.g., homozygosity for the GAG haplotype of CRHR1, can be determined using any haplotypes (genotype) analysis protocol, for example, by analysis of a sample of the subject's DNA. Any haplotypes-genotype analysis technique or protocol known in the art can be used to practice the invention, including amplification genotyping, in situ hybridization techniques, and direct DNA sequencing.

The invention also provides additional haplotype analysis to identify unique chromosomal regions containing genes predisposing an individual to disease and in studies correlating haplotypic variation with responsiveness to drug treatment, including further analysis of Mexican American, or equivalent, or other subpopulations, to determine patient responsiveness to drug therapy, including anti-depression and anti-anxiety treatment regimes and protocols. Computational methods are employed to estimate the phase and frequency of the underlying haplotypes. In this aspect, any algorithm, e.g., Expectation-Maximization (EM) algorithm, can be used to predict haplotype frequencies—usually with a high degree of accuracy.

### Generating and Manipulating Nucleic Acids

The invention provides compositions and methods comprising isolating a nucleic acid from a sample from a subject and analyzing genotype, including detecting whether the subject is homozygous for a CRHR1, or “GAG,” haplotype (homozygous for the haplotype-tag single nucleotide polymorphisms (htSNPs) rs1876828 (SEQ ID NO:2), rs242939 (SEQ ID NO:6) and rs242941 (SEQ ID NO:7). The genotype of the subject, e.g., with respect to the CRHR1, or “GAG,” haplotype, can be determined by any method or protocol or device known in the art, including amplification genotyping, in situ hybridization techniques, DNA array (biochip) analysis and/or direct DNA sequencing. Haplotypes are groups of two or more SNPs that are functionally and/or spatially linked. The invention can be practiced in conjunction with any method or protocol or device known in the art, which are well described in the scientific and patent literature.

The nucleic acids used to practice this invention, e.g., primers for use in amplification detection, sequencing, Southerns and the like, whether RNA, cDNA, genomic DNA, vectors, viruses or hybrids thereof, may be isolated from a variety of sources, genetically engineered, synthetic, amplified, and/or expressed/generated recombinantly (recombinant polypeptides can be modified or immobilized to arrays in accordance with the invention). Any recombinant expression system can be used, including bacterial, mammalian, yeast, insect or plant cell expression systems.

Alternatively, these nucleic acids can be synthesized in vitro by well-known chemical synthesis techniques, as described in, e.g., Carruthers (1982) Cold Spring Harbor Symp. Quant. Biol. 47:411-418; Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066. Double stranded DNA fragments may then be obtained either by synthesizing the complementary strand and annealing the strands together under appropriate conditions, or by adding the complementary strand using DNA polymerase with a primer sequence.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

The nucleic acids used to practice this invention, whether RNA, antisense nucleic acid, cDNA, genomic DNA, vectors, viruses or hybrids thereof, may be isolated from a variety of sources, genetically engineered, amplified, and/or expressed/generated recombinantly. Any recombinant expression system can be used, including bacterial, mammalian, yeast, insect or plant cell expression systems.

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In practicing the invention, nucleic acids (e.g., DNA from patient samples) can be detected, sequenced and/or reproduced by amplification. Amplification can also be used to sequence, clone or modify the nucleic acids of the invention. Thus, the invention provides amplification primer sequence pairs (e.g., in kits) for detecting, sequencing or amplifying nucleic acids. One of skill in the art can design amplification primer sequence pairs for any part of or the full length of these sequences.

Amplification reactions can also be used to quantify the amount of nucleic acid in a sample (such as the amount of message in a cell sample), label the nucleic acid (e.g., to apply it to an array or a blot), detect a nucleic acid or sequence (e.g., SNPs), or quantify the amount of a specific nucleic acid in a sample. In one aspect of the invention, message isolated from a cell or a cDNA library are amplified.

The skilled artisan can select and design suitable oligonucleotide amplification primers (e.g., for detecting genotype/haplotype of nucleic acid in a patient sample). Amplification methods are also well known in the art, and include, e.g., polymerase chain reaction, PCR (see, e.g., PCR Protocols, A Guide to Methods and Applications, ed. Innis, Academic Press, N.Y. (1990) and PCR Strategies (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol 152:307-316; Sambrook; Ausubel; U.S. Pat. Nos. 4,683,195 and 4,683,202; and Sooknanan (1995) Biotechnology 13:563-564.

Haplotypes (SNPs) can be detected using single-strand conformation polymorphism analysis, which identifies base differences by alteration in electrophoretic migration of single stranded PCR products, as described, e.g., U.S. Pat. No. 5,879,884; Orita et al., Proc. Nat. Acad. Sci. USA 86:2766-2770 (1989). Amplified PCR products can be generated as described above, and heated or otherwise denatured, to form single stranded amplification products. Single-stranded nucleic acids may refold or form secondary structures which are partially dependent on the base sequence. Electrophoretic mobility of single-stranded amplification products can detect base-sequence difference between alleles or target sequences.

Haplotypes (SNPs) can be detected using allele-specific PCR, which differentiates between alleles differing in the presence or absence of a variation or polymorphism. PCR amplification primers are designed to bind only to certain alleles of a target sequence; see, e.g., Gibbs (1989) Nucleic Acid Res. 17:12427-2448.

Haplotypes (SNPs) can be detected using allele-specific oligonucleotide (ASO) screening methods, e.g., as described by Saiki (1986) Nature 324:163-166. Oligonucleotides with one or more base pair mismatches are designed for any particular allele. ASO screening methods can detect variations between haplotypes. Mismatches between variant haplotypes or PCR amplified DNA can show decreased binding of the oligonucleotide relative to a variant haplotypes (or mutant) oligonucleotide. Oligonucleotide probes can be designed that under low stringency will bind to both polymorphic forms of the allele, but that at higher stringency, will bind detectably more strongly to the allele to which they correspond. Stringency conditions can be devised in which an essentially binary response is obtained, for example, an ASO corresponding to a haplotype will hybridize to that allele, and not to an alternative haplotype allele.

Haplotypes (SNPs) can be detected using ligase-mediated allele detection, e.g., as described in Landegren (1988) Science 241:1077-1080. Ligase may also be used to detect haplotypes SNPs (e.g., mutations) in a ligation amplification reaction, e.g., as described in Wu (1989) Genomics 4:560-569. The ligation amplification reaction (LAR) utilizes amplification of specific DNA sequence using sequential rounds of template dependent ligation, e.g., as in Wu (1990) Proc. Nat. Acad. Sci. USA 88:189-193.

Amplification products generated using the polymerase chain reaction can be analyzed by the use of denaturing gradient gel electrophoresis. Different haplotype alleles can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (Tm). Melting domains are at least 20 base pairs in length, and may be up to several hundred base pairs in length. Differentiation between haplotypes (SNPs) based on sequence specific melting domain differences can be assessed using polyacrylamide gel electrophoresis.

Haplotypes (SNPs) can be detected without an amplification step, based on polymorphisms including restriction fragment length polymorphisms in a patient and a family member. Hybridization probes are generally oligonucleotides which bind through complementary base pairing to all or part of a target nucleic acid. Probes can bind target sequences lacking complete complementarity with the probe sequence depending on the stringency of the hybridization conditions. Probes can be labeled directly or indirectly. By assaying for the presence or absence of the probe, the presence or absence of the target sequence can be detected. Direct labeling methods include radioisotope labeling, such as with ³²P or ³⁵S. Indirect labeling methods include fluorescent tags, biotin complexes which may be bound to avidin or streptavidin, or peptide or protein tags. Visual detection methods include photo-luminescents, Texas red, rhodamine and its derivatives, red leuco dye and 3,3′,5,5′-tetramethylbenzidine (TMB), fluorescein, and its derivatives, dansyl, umbelliferone and the like or with horse radish peroxidase, alkaline phosphatase and the like.

Haplotypes (SNPs) can be detected using an oligonucleotide ligation assay (OLA), as described, e.g., in U.S. Pat. No. 4,998,617; Landegren (1988) Science 241:1077-1080. The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. One variant nucleic acid detection assay combines attributes of PCR and OLA, see, e.g., Nickerson (1990) Proc. Natl. Acad. Sci. USA 87:8923-8927. In this method, PCR is used to achieve the exponential amplification of target DNA, which is then detected using OLA.

Haplotypes (SNPs) can be detected using a specialized exonuclease-resistant nucleotide, e.g., see U.S. Pat. No. 4,656,127. A primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

Haplotypes (SNPs) can be detected sequence-specific ribozymes, see, e.g., U.S. Pat. No. 5,498,531. This method can be used to score SNPs based on the development or loss of a ribozyme cleavage site. Perfectly matched sequences can be distinguished from mismatched sequences by nuclease cleavage digestion assays or by differences in melting temperature. If the SNP affects a restriction enzyme cleavage site, the SNP can be identified by alterations in restriction enzyme digestion patterns, and the corresponding changes in nucleic acid fragment lengths determined by gel electrophoresis.

Haplotypes (SNPs) can be detected using Genetic Bit Analysis or GBA™, e.g., see Nikiforov (1994) Nucleic Acids Res. 22(20): 4167-4175. This is a method for typing single nucleotide polymorphisms in DNA. Specific fragments of genomic DNA containing the polymorphic site(s) are first amplified by the polymerase chain reaction (PCR) using one regular and one phosphorothioate-modified primer. The double-stranded PCR product is rendered single-stranded by treatment with the enzyme T7 gene 6 exonuclease, and captured onto individual wells of a 96 well polystyrene plate by hybridization to an immobilized oligonucleotide primer. This primer is designed to hybridize to the single-stranded target DNA immediately adjacent from the polymorphic site of interest. Using the Klenow fragment of E. coli DNA polymerase I or a modified T7 DNA polymerase, the 3′ end of the capture oligonucleotide is extended by one base using a mixture of one biotin-labeled, one fluorescein-labeled, and two unlabeled dideoxynucleoside triphosphates. Antibody conjugates of alkaline phosphatase and horseradish peroxidase are then used to determine the nature of the extended base in an ELISA format.

In one aspect, haplotypes, or SNPs, are detected using biochips, or arrays. For example, several probes capable of hybridizing specifically to allelic (haplotypes) variants are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244 and in Kozal et al. (1996) Nature Medicine 2:753. In one embodiment, a chip comprises all the haplotypes (allelic) variants a gene, e.g., the corticotropin-releasing hormone receptor type 1 (CRHR1) gene and its sequence variations. The solid phase support can be contacted with a test nucleic acid and hybridization to the specific probes is detected. Thus, the identity of numerous allelic variants of one or more genes can be identified in a simple hybridization experiment.

In practicing the methods of the invention, any known array and/or method of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 21:25-32. See also published U.S. patent applications Nos. 20010018642; 20010019827; 20010016322; 20010014449; 20010014448; 20010012537; 20010008765.

Haplotypes (SNPs) can be detected using multicomponent integrated systems, such as microfluidic-based systems or “lab on a chip” systems. These systems miniaturize and compartmentalize processes such as PCR and capillary electrophoresis reactions in a single functional device. See, e.g., U.S. Pat. No. 5,589,136, which describes the integration of PCR amplification and capillary electrophoresis in chips.

Haplotypes (SNPs) can be detected using integrated systems, particularly when microfluidic systems are used. These systems can comprise a pattern of microchannels designed onto a glass, silicon, quartz, or plastic wafer included on a microchip. The movements of the samples can be controlled by electric, electroosmotic or hydrostatic forces applied across different areas of the microchip to create functional microscopic valves and pumps with no moving parts. Varying the voltage controls the liquid flow at intersections between the micro-machined channels and changes the liquid flow rate for pumping across different sections of the microchip. In such a system, the containers/compartments of the kit may be embodied as chambers and/or channels of the microfluidic system.

Haplotypes (SNPs) can be detected using mass spectrometry. Mass spectrometry takes advantage of the unique mass of each of the four nucleotides of DNA. SNPs can be unambiguously genotyped by mass spectrometry by measuring the differences in the mass of nucleic acids having alternate SNP alleles. MALDI-TOF (Matrix Assisted Laser Desorption Ionization—Time of Flight) mass spectrometry technology is preferred for extremely precise determinations of molecular mass, such as SNPs. Numerous approaches to SNP analysis have been developed based on mass spectrometry. An exemplary analysis is mini-sequencing primer extension, which can also be utilized in combination with other approaches, such as traditional gel-based formats and microarrays.

If a polymorphic region is located in an exon, either in a coding or non-coding region of the gene, the identity of the allelic variant can be determined by determining the molecular structure of the mRNA, pre-mRNA, or cDNA. The molecular structure can be determined using any of the above described methods for determining the molecular structure of the genomic DNA, e.g., sequencing and single-strand conformational polymorphism (SSCP).

Haplotype determination procedures can be performed in situ directly upon tissue sections (fixed and/or frozen) of patient tissue obtained from biopsies or resections, such that no nucleic acid purification is necessary. Nucleic acid reagents may be used as probes and/or primers for such in situ procedures

### Diagnosing and Rating Psychiatric Disorders

The invention provides methods for determining a subject's responsiveness to a psychiatric disorder therapy, wherein the subject is diagnosed with depression and anxiety. The invention also provides methods for screening a subject to determine the subject's responsiveness to a psychiatric disorder (e.g., depression and anxiety) therapy comprising use of an agent which prevents or treats a psychiatric disorder associated with the corticotropin-releasing hormone receptor 1 (CRHR1) gene. In practicing the invention any method (e.g., “rating scale”) or protocol can be used to diagnose a psychiatric disorder (e.g., depression and anxiety) or assess the progress of treatment for a psychiatric disorder (e.g., depression and anxiety).

The depression diagnosed in practicing the methods and compositions of the invention includes all diseases and conditions which are associated with depression, including those classified in the IDC-10 and Diagnostic and Statistical Manual IV (DSM-IV) rating scales. These diseases or disorders comprise major depression, dysthymic disorder, depressive episodes of bipolar disorders and depressive episodes associated with other mood disorders, including seasonal mood disorders and mood disorders due to a general medical condition and substance induced mood disorder.

For example, in practicing the invention any rating scale can be used to measure the severity of a psychiatric disorder (e.g., depression and anxiety) in a subject. For example, in depression, the most frequently used scales include the Hamilton Depression Rating (HAM-D) Scale, the Beck Depression Inventory (BDI), the Montgomery-Åsberg Depression Rating Scale (MADRS), the Geriatric Depression Scale (GDS), and the Zung Self-Rating Depression Scale (ZSRDS). For anxiety, the most frequently used scales include the Hamilton Anxiety Rating (HAM-A) Scale, and the Beck Anxiety Inventory (BAI). These or any art-acceptable means to diagnose and/or access a psychiatric disorder (e.g., depression and anxiety) in a subject can be used.

For example, in the studies described herein, the association of CRHR1 genotypes with the phenotype of antidepressant treatment response was studied in depressed Mexican-Americans who completed a prospective randomized, placebo lead-in, double-blind treatment of fluoxetine or desipramine, with active treatment for eight weeks, where the primary outcome measures of the association analysis were the changes in the Hamilton rating scales for anxiety (HAM-A) and depression (HAM-D).

The term “treatment” as used herein refers to partially or completely ameliorating at least one symptom of, partially or completely treating or curing and/or preventing the development of a disease or a condition, for example, depression or anxiety.

For example DSM-IV criteria for depression and Clinical Rating Scale for Depression are summarized below:

Major Depressive Disorder: DSM-IV Diagnostic Criteria

At least five of the following symptoms are present during the same period. At least (1) depressed mood or (2) loss of interest or pleasure must be present. Symptoms are present most of the day, nearly daily for at least 2 weeks.
1. Depressed mood (sometimes irritability in children and adolescents) most of the day, nearly every day
2. Markedly diminished interest or pleasure in almost all activities most of the day, nearly every day (as indicated either by subjective account or observation by others of apathy most of the time)
3. Significant weight loss/gain
4. Insomnia/hypersomnia
5. Psychomotor agitation/retardation
6. Fatigue (loss of energy)
7. Feelings of worthlessness (guilt)
8. Impaired concentration (indecisiveness)
9. Recurrent thoughts of death or suicide

Clinical Rating Scale for Depression
None Score=0
Meets no DSM criteria for a depressive disorder (Major depression, Dysthymia or Brief Recurrent Depression)
Has never sought help for depressive sx
Has never taken prescription meds for depressive sx
Possible Score=1
May have some depressive sx
Family members may report depressive sx
Does not meet DSM criteria for a depressive disorder
Has never sought help
Has never taken prescription medications for depression
Mild Score=2
May meet DSM criteria for a depressive disorder
May have sought help
Has never taken prescription medications for depression
Depressive symptoms were transient
Moderate Score=3
Meets DSM criteria
May have sought help
May have taken prescription medications for depression
Depressive episodes were transient but recurrent
Severe Score=4
Meets DSM criteria
Has sought help
Has usually taken prescription medications for depression
Depressive episodes have been multiple throughout life
If <20 years old, depression started by age 12
Severe and chronic Score=5
Meets all criteria for “Severe”
Has been diagnosed with depression
Depressive episodes have been chronic throughout life

### Kits

The invention provides kits suitable for determining a subject's responsiveness to a psychiatric disorder therapy. For example, kits of the invention can be used to evaluate or determine the optimal treatment, e.g., drug regimen, drug scheduling or treatment protocol, when a subject is diagnosed with depression and anxiety. The kit can comprise material for determining any particular haplotypes, e.g., whether the subject is homozygous for a CRHR1, or “GAG,” haplotype (homozygous for the haplotype-tag single nucleotide polymorphisms (htSNPs) rs1876828 (SEQ ID NO:2), rs242939 (SEQ ID NO:6) and rs242941 (SEQ ID NO:7). The kit can comprise suitable packaging material. The kit can comprise instructional material for use of said kit, e.g., instructions on practicing the methods of the invention.

The kit can comprise nucleic acids to determine a particular haplotypes or genotype, as discussed, above. For example, the kit can comprise nucleic acid that specifically binds to htSNPs, e.g., primers such as polymerase chain reaction (PCR) primers or a hybridization probes. The kit also can comprise material or items to retrieve a nucleic acid-comprising sample from a subject, and/or to store or to process the nucleic acid-comprising biological sample. The kits comprise a vial, tube, or any other container which contains one or more oligonucleotides or primers which hybridize to a nucleic acid isolated form a subject, or a nucleic acid derived from a subject, e.g., an amplification product. The kits may also contain components of the amplification system, including PCR reaction materials such as buffers and a thermostable polymerase. A kit of the invention can be used in conjunction with commercially available amplification kits, e.g., from GIBCO BRL (Gaithersburg, Md.) Stratagene (La Jolla, Calif.), Invitrogen (San Diego, Calif.), Schleicher & Schuell (Keene, N.H.), Boehringer Mannheim (Indianapolis, Ind.). A kit of the invention also can comprise positive or negative control reactions or markers, molecular weight size markers for gel electrophoresis, and the like. A kit of the invention also can comprise labeling or instructions indicating the suitability of the kits for diagnosing depression and indicating how the oligonucleotides are to be used for that purpose.

### Corticotropin-Releasing Hormone Receptor 1 (CRHR1) Protein or Transcript Isoforms

The invention provides methods for determining a corticotropin-releasing hormone receptor 1 (CRHR1) protein or transcript isoform associated with a specific response to a psychiatric disorder therapy (e.g., any CRHR1-associated psychiatric disorder, such as depression, anxiety, or anxiety and depression) in an individual comprising (a) determining the CRHR1 protein or transcript isoform or isoforms expressed in the individual, comparing the CRHR1 protein or transcript isoform or isoforms expressed in the individual to CRHR1 protein or transcript isoform or isoforms expressed in individuals having the same psychiatric disorder therapy; and, (b) correlating the response of the individual to the psychiatric disorder therapy with the presence or absence of the expressed CRHR1 protein or transcript isoform or isoforms, wherein optionally the psychiatric disorder therapy is a drug therapy, e.g., for depression and/or anxiety. The results of this method can be used by a clinician to determine what therapy or drugs to use, in what combinations and/or in what dosages, or for what patient populations.

The invention provides methods for determining a corticotropin-releasing hormone receptor 1 (CRHR1) protein or transcript isoform associated with the presence of a psychiatric disorder (e.g., e.g., any CRHR1-associated psychiatric disorder, such as depression, anxiety, or anxiety and depression) in an individual comprising (a) determining the CRHR1 protein or transcript isoform or isoforms expressed the individual, comparing the CRHR1 protein or transcript isoform or isoforms expressed in the individual to CRHR1 protein or transcript isoform or isoforms expressed in individuals having the same psychiatric disorder, and determining the presence or absence of CRHR1 protein or transcript isoform or isoforms in individuals having the same psychiatric disorder; and, (b) correlating the diagnosis of the psychiatric disorder to the presence or absence of the CRHR1 protein or transcript isoform or isoforms expressed in the individuals, wherein optionally the psychiatric disorder therapy is a depression and/or anxiety therapy.

The invention provides methods for diagnosing the presence of a psychiatric disorder (e.g., e.g., any CRHR1-associated psychiatric disorder, such as depression, anxiety, or anxiety and depression) in an individual by determining what corticotropin-releasing hormone receptor 1 (CRHR1) or corticotropin-releasing hormone receptor 2 (CRHR2) protein or transcript isoforms are expressed in an individual comprising (a) determining the CRHR1 or CRHR2 protein or transcript isoform or isoforms expressed the individual, comparing the CRHR1 or CRHR2 protein or transcript isoform or isoforms expressed in the individual to CRHR1 or CRHR2 protein or transcript isoform or isoforms expressed in individuals having the same psychiatric disorder, and determining the presence or absence of CRHR1 or CRHR2 protein or transcript isoform or isoforms expressed in individuals having the same psychiatric disorder; and, (b) correlating the presence or absence of the CRHR1 or CRHR2 protein or transcript isoform or isoforms expressed in the individuals to the presence or absence of a psychiatric disorder, wherein optionally the psychiatric disorder therapy is a depression and/or anxiety therapy. The results of this method can also be used in the prognosis of a psychiatric disorder or predicting the outcome of therapy for a psychiatric disorder, e.g., a therapy for depression and/or anxiety.

Determining CRHR1 or CRHR2 protein or transcript isoform or isoforms can be accomplished using any method or protocol, e.g., as described herein, all of which are well known in the art, including, e.g., PCR or other amplification protocols, electrophoresis molecular sizing, antibodies specific for particular alternatively spliced protein motifs, and the like.

Alternatively expressed CRHR1 or CRHR2 protein or transcript isoform or isoforms have been described, for example, by Pisarchik (2001) The FASEB Journal 15:2754-2756, who note that human and mouse CRH-R1e isoforms contain two reading frames, of which one encodes soluble proteins of 194 aa in humans and 139 aa in mice containing the first 40 aa of distal amino-terminal sequence with a remaining sequence different from the CRH-R1 receptor due to the frameshift. Because of the lack of exons 3, 4 and transmembrane domains, it should not act as a CRH binding protein. The second form (human: 240 aa; mouse: 309 aa) with a sequence starting from the third transmembrane domain in humans and first transmembrane domain in the mouse contains the carboxyl terminus. It will not be able to bind a ligand because of the lack of an NH₂ terminus. Human CRH-R1f encodes an entire CRH binding domain and the first five transmembrane domains; therefore, it should bind CRH and fix it on the outer surface of cellular membrane. It may thus decrease local concentration of CRH or serve as a pool of bound hormone. The murine form of this receptor also encodes the entire NH₂ terminus and five transmembrane domains. CRH-R1g, in which the reading frame was preserved but the protein sequence had a deletion of 74 amino acids corresponding to transmembrane domains 5 and 6, can potentially be coupled to cAMP production. CRH-R1h encodes a truncated protein having only a CRH binding domain and can potentially interfere with CRH binding or serve as an analog of CRH binding protein.

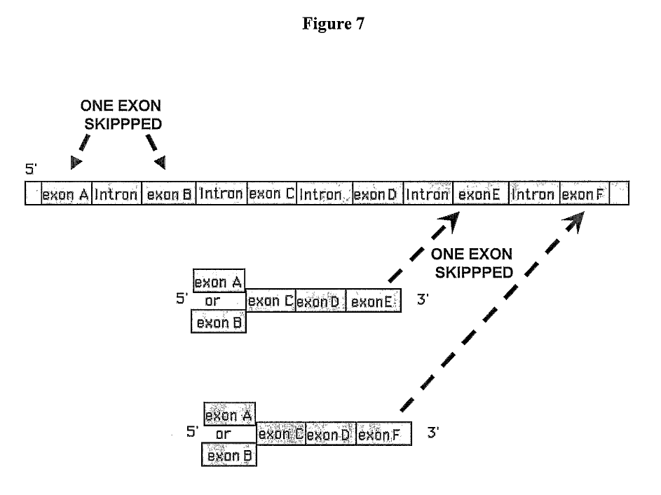

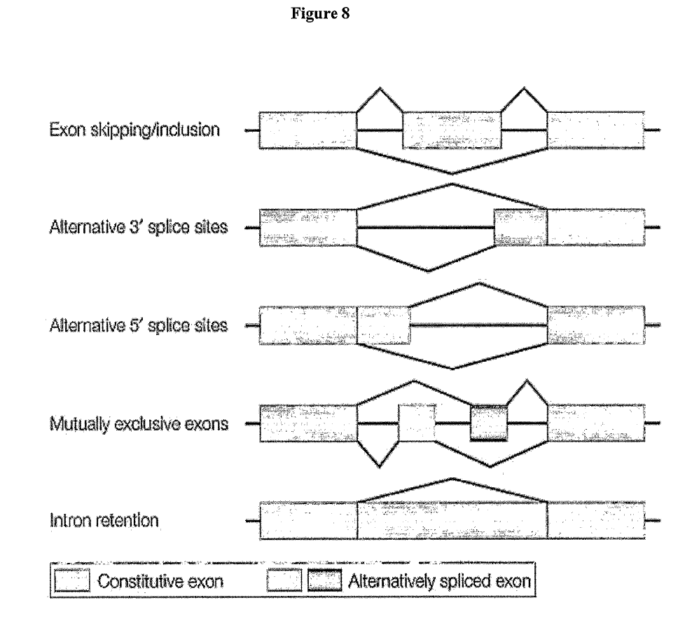

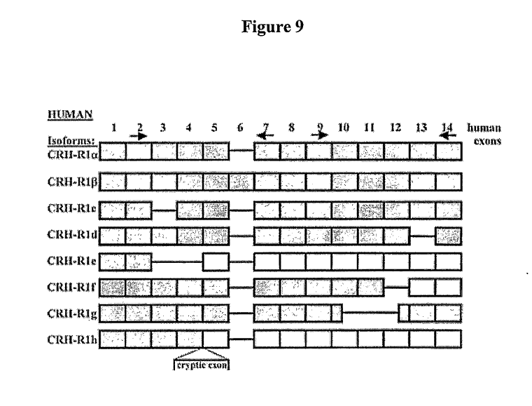

See also FIGS. 7, 8 and 9, illustrating alternative human CRH-R1 transcript forms generated by alternative splicing events. These alternative human CRH-R1 isoforms can serve as guidance to determine which human CRH-R1 transcript or protein isoforms are associated with a particular psychiatric disorder therapy, but are not limiting as to what CRH-R1 transcript or protein isoforms can be used or may be found using the methods of the invention.

While the invention is not limited by any particular mechanism of action, because the stress-induced HPA axis activation is thought to directly cause depressive symptoms by interacting with the brain neurotransmitter systems regulating these behavioural changes, the methods of the invention also comprise methods for associating a protein isoform or transcript isoform with a specific response to a psychiatric disorder therapy in an individual, wherein the protein is involved in the hypothalamic-pituitary-adrenal (HPA) axis pathway. In one aspect, the methods comprise (a) determining the protein or transcript isoform or isoforms expressed in the individual, comparing the protein or transcript isoform or isoforms expressed in the individual to protein or transcript isoform or isoforms expressed in individuals having the same psychiatric disorder therapy; and, (b) correlating the response of the individual to the psychiatric disorder therapy with the presence or absence of the expressed protein or transcript isoform or isoforms, wherein in one aspect the psychiatric disorder therapy is a drug therapy, and in one aspect the protein is involved in the hypothalamic-pituitary-adrenal (HPA) axis pathway is corticotropin-releasing hormone (CRH), CRHR1 or CRHR2, proopiomelanocortin (POMC), urocortin (UCN), stresscopin (UCN3), stresscopin related peptide (UCN2), a protein involved in steroid synthesis or degradation, or a transcription factor involved in the HPA axis pathway.

This aspect is further supported by clinical studies showing that normalization of HPA activity by antidepressants precedes the therapeutic effects on the depressive symptoms. While the invention is not limited by any particular mechanism of action, two pathways by which the activation of the HPA axis can participate in the development of depression include: elevated levels of cortisol induce the depressive symptoms (e.g., as in Cushing's disease); and, the lack of effects of cortisol induces the depressive symptoms (e.g., as in Addison's disease). Thus, in one aspect, the invention provides methods for associating a protein isoform or its transcript isoform with a specific response to a psychiatric disorder therapy in an individual, wherein the protein is involved in the hypothalamic-pituitary-adrenal (HPA) axis pathway, comprising (a) determining the protein or transcript isoform or isoforms expressed in the individual, comparing the protein or transcript isoform or isoforms expressed in the individual to protein or transcript isoform or isoforms expressed in individuals having the same psychiatric disorder therapy; and, (b) correlating the response of the individual to the psychiatric disorder therapy with the presence or absence of the expressed protein or transcript isoform or isoforms, wherein in one aspect the psychiatric disorder therapy is a drug therapy, and in one aspect the protein is involved in the hypothalamic-pituitary-adrenal (HPA) axis pathway is corticotropin-releasing hormone (CRH), CRHR1 or CRHR2, proopiomelanocortin (POMC), urocortin (UCN), stresscopin (UCN3), stresscopin related peptide (UCN2), a protein involved in steroid synthesis or degradation, or a transcription factor involved in the HPA axis pathway.

The invention also provides methods for associating a protein isoform or its transcript isoform associated with the presence of a psychiatric disorder in an individual, wherein the protein is involved in the hypothalamic-pituitary-adrenal (HPA) axis pathway, comprising (a) determining the protein or transcript isoform or isoforms expressed the individual, comparing the protein or transcript isoform or isoforms expressed in the individual to protein or transcript isoform or isoforms expressed in individuals having the same psychiatric disorder, and determining the presence or absence of protein or transcript isoform or isoforms in individuals having the same psychiatric disorder; and, (b) correlating the diagnosis of the psychiatric disorder to the presence or absence of the protein or transcript isoform or isoforms expressed in the individuals, wherein in one aspect the psychiatric disorder therapy is a depression and in one aspect the protein is involved in the hypothalamic-pituitary-adrenal (HPA) axis pathway is corticotropin-releasing hormone (CRH), CRHR1 or CRHR2, proopiomelanocortin (POMC), urocortin (UCN), stresscopin (UCN3), stresscopin related peptide (UCN2), a protein involved in steroid synthesis or degradation, or a transcription factor involved in the HPA axis pathway.

The invention also provides methods for diagnosing the presence of a psychiatric disorder in an individual by determining what protein or transcript isoforms are expressed in an individual wherein the protein is involved in the hypothalamic-pituitary-adrenal (HPA) axis pathway, comprising (a) determining the protein or transcript isoform or isoforms expressed the individual, comparing the protein or transcript isoform or isoforms expressed in the individual to protein or transcript isoform or isoforms expressed in individuals having the same psychiatric disorder, and determining the presence or absence of protein or transcript isoform or isoforms expressed in individuals having the same psychiatric disorder; and, (b) correlating the presence or absence of the protein or transcript isoform or isoforms expressed in the individuals to the presence or absence of a psychiatric disorder, wherein optionally the psychiatric disorder therapy is a depression and optionally the protein is involved in the hypothalamic-pituitary-adrenal (HPA) axis pathway is corticotropin-releasing hormone (CRH), CRHR1 or CRHR2, proopiomelanocortin (POMC), urocortin (UCN), stresscopin (UCN3), stresscopin related peptide (UCN2), a protein involved in steroid synthesis or degradation, or a transcription factor involved in the HPA axis pathway.

The invention will be further described with reference to the following examples; however, it is to be understood that the invention is not limited to such examples.

### EXAMPLES

### Example 1

### Predicting Patient Responses to Antidepressants

The following example describes making and using exemplary compositions of the invention and how to practice the compositions and methods of the invention. The following example also describes studies and data demonstrating that the compositions and methods are effective for predicting the response of a sub-population of patients (e.g., Mexican-Americans) to antidepressants and to aid in monitoring, evaluating and adjusting an on-going regimen of antidepressant drug therapy. In particular, these studies demonstrate that the compositions and methods of the invention can be used to predict and monitor treatment responses in a phenotypic subgroup of depressed patients, a high-anxiety depressed patient subpopulation, by determining the presence, or absence, of homozygozity for the GAG haplotypes of the corticotropin-releasing hormone receptor type 1 (CRHR1) gene. Stratification of treatment response to antidepressant drugs was observed in a phenotypic subgroup of high-anxiety depressed patients according to a haplotype of CRHR1.

The association of CRHR1 genotypes with the phenotype of antidepressant treatment response was studied in 80 depressed Mexican-Americans in Los Angeles who completed a prospective randomized, placebo lead-in, double-blind treatment of fluoxetine or desipramine, with active treatment for eight weeks. While the invention is not limited to a particular ethnic group, the analyses reported herein were confined to Mexican-Americans, to minimize concerns about possible population stratification. The primary outcome measures of the association analysis were the changes in the Hamilton rating scales for anxiety (HAM-A) and depression (HAM-D).

Because CRHR1 antagonists are effective for amelioration of both depressive symptoms and anxiety-like behaviors, the phenotype studied was refined to differentiate depressed subjects into two groups: high and low anxiety, as defined by their scores on the HAM-A, a highly validated rating scale for anxiety. It was hypothesized that variants of the CRHR1 gene would be more likely to be associated with treatment responses in a subgroup of patients who met diagnostic criteria for a current episode of major depression and who were also highly anxious.

SNPs in the CRHR1 gene were genotyped via a SEQUENOM MASSARRAY™ MALDI-TOF mass spectrometer (Sequenom, San Diego, Calif., USA) for analysis of unlabeled single-base extension minisequencing reactions with a semiautomated primer design program (SPECTRODESIGNER™, Sequenom). The protocol implemented the very short extension method, whereby sequencing products are extended by only one base for three of the four nucleotides and by several additional bases for the fourth nucleotide (representing one of the alleles for a given SNP), permitting clearly delineated mass separation of the two allelic variants at a given locus. Nine SNPs were assayed in CRHR1 corresponding to the following dbSNP identifiers: rs171440 (SEQ ID NO:1), rs1876828 (SEQ ID NO:2), rs1876829 (SEQ ID NO:3), rs1876831 (SEQ ID NO:4), rs242938 (SEQ ID NO:5), rs242939 (SEQ ID NO:6), rs242941 (SEQ ID NO:7), rs242949 (SEQ ID NO:8), rs242950 (SEQ ID NO:9).

Haplotype frequencies with respect to the CRHR1 gene were imputed using EM algorithm-based estimation routines implemented in the S-PLUS™ software package HAPLO.STATS Version 1.1.0™ on the entire group of patient and control subjects; haplotypes were assigned to an individual based upon maximal posterior probabilities. As a person can have more than one haplotype, the number and type of haplotypes for each subject was counted. A haplotype variable was created that had a value of 0, 1, or 2 for the count of haplotypes. Nine SNPs, spanning 27 kb of the CRHR1 gene, were successfully genotyped in both the patient and control groups. Subsequently, a haplotype-tag approach was used to identify a parsimonious set of SNPs, called haplotype tagging SNPs (htSNPs) with 5% or greater frequency. A minimal subset of htSNPs, capable of distinguishing the haplotypic variations within a population, was selected that was identical for both patients and controls. These SNPs were tested for haplotype association using PROC GLM™ in SAS program (SAS, version 8 (Cary, N.C., USA).

Subjects were diagnosed according to the Structured Clinical Interview for DSM-IV (SCID) and included into the study if they had a diagnosis of depression without other confounding medical or psychiatric diagnoses or treatments. All patients were followed weekly and assessed for changes in the Hamilton rating scales for anxiety (HAM-A) and depression (HAM-D). Inclusion criteria in the study included a HAM-D of 18 or higher. Patients were classified into a high anxiety (HA) group if their HAM-A score was 18 or higher and in a low anxiety (LA) group if their HAM-A score was less than 18.

Stratification of treatment response to antidepressant drugs was observed in a phenotypic subgroup of high-anxiety depressed patients according to a haplotype of CRHR1. Utilizing the haplotype-tag single nucleotide polymorphisms (htSNPs) identified as rs1876828 (SEQ ID NO:2), rs242939 (SEQ ID NO:6) and rs242941 (SEQ ID NO:7), haplotypic association between CRHR1 and eight-week response to daily antidepressant treatment was determined. In the HA group (n=54), homozygozity for the GAG haplotype was associated with a relative 70% greater reduction in HAM-A scores compared to heterozygous (63.1±4.5% versus 37.1±6.9%, respectively, P=0.002). For HAM-D, GAG haplotype homozygozity was associated with a 31% greater reduction in scores after treatment compared to heterozygous (67.3±4.3% versus 51.2±6.0%, respectively, P=0.03). In those with lower anxiety levels at screening there were no associations between CRHR1 genotype and % change in HAM-A or HAM-D.

Significant association of antidepressant treatment response and a CRHR1 haplotype (presence of haplotype-tag single nucleotide polymorphisms (htSNPs) identified as rs1876828 (SEQ ID NO:2), rs242939 (SEQ ID NO:6) and rs242941 (SEQ ID NO:7) occurred only in the high anxiety group. The association was not present in the low anxiety group or when low and high anxiety subjects were grouped together. These results suggest that the aggregation of all patients undergoing a treatment into one group may limit the ability to detect the effects of genetic variants on pathophysiologically defined subgroups. The findings of increased response to antidepressants in highly anxious patients homozygous for the GAG haplotype of CRHR1 support the hypotheses that response to antidepressant treatment is heterogeneous and that the CRHR1 gene and possibly other genes in stress-inflammatory pathways are involved in the response to antidepressant treatment. These findings also demonstrated that CRHR1 gene variation may affect response to CRHR1 agonists or antagonists. CRHR1 haplotypes were not stratified by diagnosis of depression and were similarly distributed in depressed and control subjects.

The findings also demonstrated that chromosome 17q11-q22 is a hot spot for genes implicated in antidepressant response, as the CRHR1 and the serotonin transporter gene which has already been implicated in the antidepressant response are located in chromosome 17q11-q22. The findings also demonstrated that inflammatory pathways and chromosome 17q11-q22 are involved in the pathophysiology of major depression. Thus, the invention provides a method for method for determining a nucleotide polymorphism associated with a response (e.g., a positive response, a negative response, no response, a side effect, a dosage response, and the like) to a psychiatric disorder therapy in an individual comprising (a) sequencing all or a portion of the nucleotide sequence of chromosome 17q11-q22 from the individual, comparing the 17q11-q22 sequence from the individual to chromosome 17q11-q22 sequences of other individuals, and determining the presence of a nucleotide polymorphism in the sequenced 17q11-q22 sequences; and, (b) correlating the response of the individual to a psychiatric disorder therapy with the presence or absence of the nucleotide polymorphism, wherein optionally the psychiatric disorder therapy is a drug therapy.

SNP Sequences

SEQ ID NO:1

rs171440
TCCCCTGCTCTGTAGCCTAAGGACA[C/T]TTCTCTTGGTCCCTCGCATG
GTGAC

SEQ ID NO:2

rs1876828
GCAGCATACCCCTAGGGACCTAGGA[A/G]CAGGGAGGGAGAGAGGCAGC
CCTGG

SEQ ID NO:3

rs1876829
GTCCCAACAGGCCTCACAGCCCTGA[A/G]CCCCGCTGCAGGGCCCCCGG
GTCCT

SEQ ID NO:4

rs1876831
CCCCAACCAGAGATGATGATGGGGG[A/G]CAGGGGAGGCACCAAACCCT
GGGCC

SEQ ID NO:5

rs242938
CCTTTCCTGGGATCACAGAGGGAAG[C/T]GCGGGGGAGCCTAGAGAGCA
CCACA

SEQ ID NO:6

rs242939
GAACACGGAGGCCACACAAGAGTGG[A/G]TTCCAAGTGAAGGAGTGACC
AACTC

SEQ ID NO:7

rs242941
GGGCCAGGAACCATGAACCAGCGCG[G/T]GTGGGGGCAGCCTCTTCAGG
CCTGG

SEQ ID NO:8

rs42949
TGCCATGAAGGCTCCCTGCCCTCAA[A/C]CCACATGTATCCCCTGCCCA
GGGCT

SEQ ID NO:9

rs242950
GGTGGCCCCCACCTCTAGGTAGAGG[A/G]GTCCTTTCTGTCCACGGTTG
GCACT
htSNPs - GAG

SEQ ID NO:10

rs1876828(G)
GCAGCATACCCCTAGGGACCTAGGA[G]CAGGGAGGGAGAGAGGCAGCCC
TGG

SEQ ID NO:11

rs242939(A)
GAACACGGAGGCCACACAAGAGTGG[A]TTCCAAGTGAAGGAGTGACCAA
CTC

SEQ ID NO:12

rs242941(G)
GGGCCAGGAACCATGAACCAGCGCG[G]GTGGGGGCAGCCTCTTCAGGCC
TGG

### The Study Population

The study population consisted of 233 depressed subjects enrolled in an ongoing pharmacogenetic study of antidepressant treatment response to desipramine or fluoxetine. Data from the first 80 subjects who completed the protocol and for whom we had available genetic data are reported herein. Also studied were 251 age- and sex-matched control subjects who were recruited from the same Mexican-American community in Los Angeles, and studied by the same, bilingual, clinical research team at the Center for Pharmacogenomics and Clinical Pharmacology, Neuropsychiatric Institute, David Geffen School of Medicine at UCLA. Controls were in general good health but were not screened for medical or psychiatric illness, to avoid bias. All patients are Mexican-American men and women ages 21-68, with a current episode of major depression as diagnosed by the DSM-IV. In this study, all Mexican-American subjects had at least three grandparents born in Mexico. We used diagnostic and rating instruments that have been fully validated in English and in Spanish, and conducted all assessments in the subjects' primary language.

Inclusion criteria included DSM-IV diagnosis of current, unipolar major depressive episode, with a 21-Item Hamilton Depression Rating Scale (HAM-D) score of 18 or greater with item number 1 (depressed mood) rated 2 or greater. There was no anxiety threshold for inclusion. Subjects with any primary axis I disorder other than major depressive disorder (e.g. dementia, psychotic illness, bipolar disorder, adjustment disorder), electroconvulsive therapy in the last six months, or previous lack of response to desipramine or fluoxetine were excluded. Because anxiety can be a manifestation of depression, patients who met criteria for depression and also anxiety disorders were not excluded. Exclusion criteria included active medical illnesses that could be etiologically related to the ongoing depressive episode (e.g., untreated hypothyroidism, cardiovascular accident within the past six months, uncontrolled hypertension or diabetes), current, active suicidal ideation with a plan and strong intent, or recent history of a serious suicide attempt, pregnancy, lactation, current use of medications with significant central nervous system activity which interfere with EEG activity (e.g. benzodiazepines) or any other antidepressant treatment within the 2 weeks prior to enrollment, illicit drug use and/or alcohol abuse in the last three months or current enrollment in psychotherapy.

All patients had an initial comprehensive psychiatric and medical assessment, and, if enrolled, had 9 weeks of structured follow-up assessments. The study consisted of two phases: a one-week, single-blind placebo lead-in phase to eliminate placebo responders, followed, if subjects continue to meet inclusion criteria after phase 1, by random assignment to one of the two treatment groups: fluoxetine 10 mg-40 mg/day or desipramine 50 mg-200 mg/day, administered in a double-blind fashion for 8 weeks, with dose escalation based on clinical outcomes. The study population consisted of the first 80 subjects who completed the trial, with weekly data collection, and for whom we obtained genotype data. The study used a combined sample of patients taking the selective serotonin reuptake inhibitor, fluoxetine, or the tricyclic antidepressant, desipramine, because both drugs have been shown to have equal effects on downregulating CNS CRH gene expression. It is consequently justifiable to group together patients taking desipramine and imipramine for the purposes of studying SNPs in this gene, because both of these drugs have effects on CRH function as a common, final pathway of action. Moreover, in our ongoing clinical study, no differences between the two drugs in terms of antidepressant effectiveness have been observed, which is consistent with findings from the literature.

### SNP Genotyping Methods

As described above, 9 SNPs were assayed in CRHR1 corresponding to the following dbSNP identifiers: rs171440 (SEQ ID NO:1), rs1876828 (SEQ ID NO:2), rs1876829 (SEQ ID NO:3), rs1876831 (SEQ ID NO:4), rs242938 (SEQ ID NO:5), rs242939 (SEQ ID NO:6), rs242941 (SEQ ID NO:7), rs242949 (SEQ ID NO:8), rs242950 (SEQ ID NO:9). The SNPs were genotyped via a SEQUENOM MassARRAY MALDI-TOF mass spectrometer (Sequenom, San Diego, Calif., USA) for analysis of unlabeled single-base extension minisequencing reactions with a semiautomated primer design program (SpectroDESIGNER, Sequenom). The protocol implemented the very short extension method, whereby sequencing products are extended by only one base for three of the four nucleotides and by several additional bases for the fourth nucleotide (representing one of the alleles for a given SNP), permitting clearly delineated mass separation of the two allelic variants at a given locus.

### Haplotype Scoring Methods

Haplotype frequencies were imputed using EM algorithm-based estimation routine implemented in the S-PLUS™ software package HAPLO.STATS Version 1.1.0™ on the entire group of depressed and control subjects; haplotypes were assigned to an individual based upon maximal posterior probabilities. As a person can have more than one haplotype, the number and type of haplotypes for each subject was counted. A haplotype variable was created that had a value of 0, 1, or 2 for the count of haplotypes. Nine (9) SNPs spanning 27 kb of the CRHR1 gene were successfully genotyped in both the depressed and control groups. Subsequently, we used a haplotype-tag approach to identify htSNPs for haplotypes with 5% of greater frequency. We chose a minimal subset of htSNPs that was identical for both depressed and controls. These SNPs were tested for haplotype association using proc GLM in SAS program (SAS, version 8 (Cary, N.C., USA).

Hardy Weinberg Equilibrium: The Hardy Weinberg equation was used to test for differences between the actual and expected frequencies of individual SNPs within haplotypes of interest.

### Statistical Methods

The primary phenotypic outcome measure HAM-D was converted to percent change in HAM-D. The percent change in HAM-D was defined as:

$$\frac{HAM \text{-} {D}_{wk 0} - HAM \text{-} {D}_{wk 8}}{HAM \text{-} {D}_{wk 0}} \times 100$$

Similarly, the percent change in HAM-A was defined as:

$$\frac{HAM \text{-} {A}_{wk 0} - HAM \text{-} {A}_{wk 8}}{HAM \text{-} {A}_{wk 0}} \times 100.$$

Eighty subjects were divided into a low anxiety group (LA) with HAM-A score less than 18 at screening and a high anxiety group (HA), with HAM-A scores that were 18 or higher at screening. Inclusion criteria into the study included a HAM-D of 18 or higher. Therefore HA subjects had HAM-D and HAM-A scores that were 18 or higher and LA subjects had a HAM-D that was 18 or higher and HAM-A score that was less than 18. Associations between haplotypes and antidepressant response were tested using generalized linear models (Proc GLM in SAS) under the assumption of an additive model. Demographics of the LA group and HA group were assessed for homogeneity using χ² or t-tests, as appropriate.

### Results

Of 233 depressed patients who were genotyped, 198 had no missing data in the SNPs of interest, representing a genotype success rate of 85%. Of the 198 genotyped subjects, 80 completed the treatment trial and were included in this analysis. Among those 80 patients, there were 54 subjects in the HA group and 26 subjects in the LA group. Clinical and demographic variables are shown in Table 1, below:

TABLE 1
Clinical and demographics characteristics of the antidepressant-treated sample:

| Diagnosis | controls | depressed | depressed | P value* |
|---|---|---|---|---|
| Anxiety status | | Low anxiety | High anxiety | |
| | | (HAM-A <18) | (HAM-A >=18) | |
| Number | 251 | 26 | 54 | |
| Sex (female/male) | (159/92) | (18/8) | (40/14) | 0.64 |
| Age (mean/stdev) | | 42.1(10.8) | 39.6(10.6) | 0.326 |
| Treatment | | (16/10) | (22/32) | 0.08 |
| (Desipramine/Fluoxetine) | | | | |
| Non-Responder/Responder | | (9/17) | (13/41) | 0.29 |
| Acculturation (mean/stdev) | | 39/17 | 31/8 | 0.09 |
| HAM-D score at wk 0 | | 19.6(1.9) | 23(4.2) | 0.0001 |
| Haplotype 1 (1 copy/2 copies) | (79/83) | (16/10) | (23/31) | 0.11 |

By χ² or t-test.

χ² tests of sex, treatment, number of copies of haplotype 1 (GAG, see table 2), and responder status showed no difference between HA and LA groups. HA and LA groups were also similar in age (P=0.326) and acculturation score (P=0.9). As expected the average HAM-D score at week 0 of 23.0±4.2 in HA group was significantly higher (P=0.0001) than in the LA group (19.6±1.9). Haplotype frequencies were estimated using HAPLO.SCORE™ (a suite of routines that can be used to compute score statistics to test associations between haplotypes and a wide variety of traits, including binary, ordinal, quantitative, and Poisson; Rowland, et al., Mayo Foundation for Medical Education and Research), as shown in Table 2, below:

TABLE 2
Haplotype frequencies estimated by HAPLO.SCORE ™.

| | rs1876828 | rs242939 | rs242941 | All | |
|---|---|---|---|---|---|
| | (SEQ ID | (SEQ ID | (SEQ ID | depressed | Controls |
| Haplotype | NO: 2) | NO: 6) | NO: 7) | (n = 233) | (n = 251) |
| 1 | G | A | G | .63197 | 0.66327 |
| 2 | G | A | T | .17930 | 0.16281 |
| 3 | A | A | G | .12218 | 0.12983 |
| 4 | G | G | T | .06655 | 0.04409 |

No difference in the frequencies of haplotypes in depressed and control groups (P = 0.9).

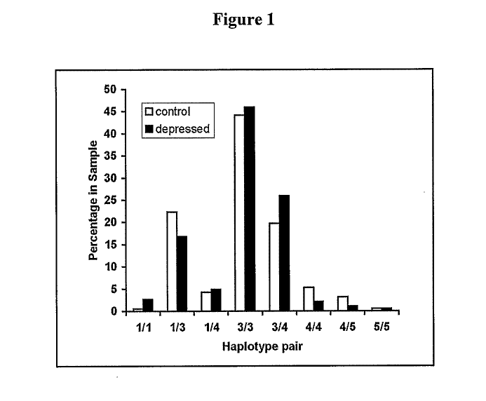

No difference was found in the frequencies of haplotypes in depressed and control groups (P=0.9). Four common haplotypes comprised of 99.6% of the total haplotypic substructure for both depressed and control groups. The frequency of those haplotypes/subject in our population of all depressed and all controls is shown in FIG. 1 (which illustrates the frequencies of CRHR1 haplotypes/subject in a population of 233 depressed and 251 controls).

Utilizing the htSNPs rs1876828 (SEQ ID NO:2), rs242939 (SEQ ID NO:6) and rs242941 (SEQ ID NO:7), we analyzed the rate of HAM-A and HAM-D improvements in patients with the GAG haplotype (haplotype 1), which had an allele frequency of 0.63197 in depressed and 0.66327 in controls (non-significant). Subjects' eight-week responses to daily antidepressant treatment (fluoxetine or desipramine) were stratified by CRHR1 GAG haplotype status, utilizing the htSNPs rs1876828 (SEQ ID NO:2), rs242939 (SEQ ID NO:6) and rs242941 (SEQ ID NO:7). In this group of 80 subjects there were no individuals with zero copies of haplotype 1; therefore, patients were classified as being either homozygous (two (2) copies of this haplotype) or heterozygous (one (1) copy of this haplotype).

In the HA group (n=54), those who were homozygous for the GAG haplotype had a decrease in HAM-A of 63.1±4.5% (average±sem); in those heterozygous for the GAG haplotype this rate was 37.1±6.9%, (P=0.002). Thus, homozygozity for the GAG haplotype was associated with a relative 70% greater reduction in HAM-A scores in comparison to heterozygous.

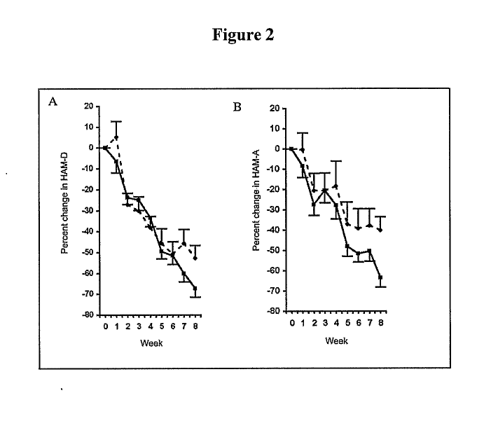

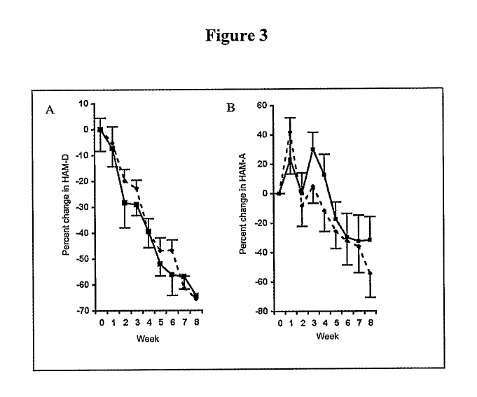

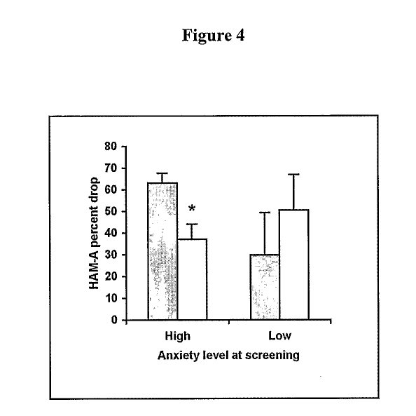

In those with lower anxiety levels at screening (n=26), HAM-A scores decreased 29.9±6.2% in patients homozygous for haplotype 1 and 50.5±4.1% in those who were heterozygous for that haplotype (P=0.43, non-significant), as illustrated in FIG. 2, FIG. 3 and FIG. 4.

FIGS. 2a and 2b illustrate data showing the percent decrease in HAM-D, FIG. 2(a), and HAM-A, FIG. 2(b), across 8 weeks of double-blind antidepressant treatment in 54 depressed Mexican-Americans with high levels of anxiety (HAM-A scores of 18 or higher). Those who were homozygous for haplotype 1 (defined by GAG at htSNPs rs1876828 (SEQ ID NO:2), rs242939 (SEQ ID NO:6) and rs242941 (SEQ ID NO:7)) of the CRHR1 gene had significantly greater responses than those who were heterozygous, with one copy of that haplotype (P=0.002 for HAM-D [a] P=0.03 for HAM-A [b].

FIGS. 3a and 3b illustrate data showing the percent decrease in HAM-D FIG. 3(a), and HAM-A, FIG. 3(b), across 8 weeks of double-blind antidepressant treatment in 26 depressed Mexican-Americans with lower levels of anxiety (HAM-A score lower than 18). There were no significant differences in HAM-D or HAM-A scores in patients who were homozygous or heterozygous for CRHR1 haplotype 1 (GAG).

FIG. 4 illustrates data of an eight-week response to daily antidepressant treatment (fluoxetine or desipramine), stratified by CRHR1 GAG haplotype, calculated utilizing the htSNPs rs1876828 (SEQ ID NO:2), rs242939 (SEQ ID NO:6) and rs242941 (SEQ ID NO:7). The mean % in HAM-A decrement in highly anxious (HA), depressed patients with the GAG/GAG homozygous haplotype was 70% greater than in those who were heterozygous for this haplotype (left-side bars). In patients with lower anxiety levels (LA) at screening (right-side bars), there was no significant difference in the % decrease of HAM-A scores in homozygous for haplotype 1 compared to heterozygous. Bars represent mean ±sem; * indicates P=0.002.

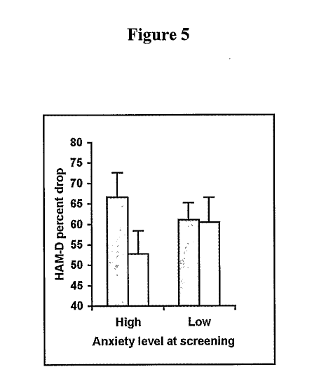

FIG. 5 illustrates an eight-week response to daily antidepressant treatment (fluoxetine or desipramine), as assessed by % decrement in HAM-D scores, stratified by GAG haplotype in depressed Mexican-Americans. The mean % HAM-D score decrease in HA patients with the GAG/GAG homozygous haplotype was 31% greater than in heterozygous (left-side bars, P=0.03). In LA patients (right-side bars), reduction in HAM scores during the course of antidepressant treatment was not significantly different between CRHR1 haplotype 1 homozygous and heterozygous. Bars represent mean ±sem; * indicates P=0.03.

Likewise, in the HA group, homozygozity for the GAG haplotype was associated with a significant decrease in HAM-D of 67.3±4.3%; in those heterozygous for the GAG haplotype the rate of decrease was significantly lower: 51.2±6.0%, (P=0.03). This represents a relative 31% greater reduction in scores after treatment in homozygous compared to heterozygous.

In those with lower anxiety levels at screening, HAM-D scores decreased 61.0±1.9% in those homozygous for haplotype 1 and 60.5±1.4% in heterozygous (P=0.95, non-significant).

FIG. 4 summarizes the findings of a significant % decrease in HAM-A in antidepressant-treated patients stratified by level of anxiety and CRHR1 genotype (this association was also significant for the phenotype of % change in HAM-D). In the LA group there no significant association between number of copies of haplotype 1 and percent (%) decrease in the HAM-A (or HAM-D). There was no difference between drugs in the association of clinical effects and CRHR1 genotype in this sample.

The genotype results at each htSNP were examined to determine whether specific SNPs were driving the association of treatment response with haplotype 1. There was no association between each SNP and decreases in HAM-A or HAM-D in the entire group of 80 depressed (P=0.2). In the HA subgroup, we found non-significant trends for association of treatment response and individual SNPs: in htSNP rs1876828A1 (SEQ ID NO:2) there was a trend for a difference (P=0.09) in the rate of HAM-A decrease in those with the G allele versus the A allele (56.0±4.6% and 38.2±9.6%, respectively). The frequency of the G allele was 42/52 and the A allele was 10/52.

Genotyping of htSNP rs1876828A2 (SEQ ID NO:2) showed G in the entire group of 80 subjects. In htSNP rs242939 (SEQ ID NO:6) there was variation in the group of treated depressed subjects, who all had A (see table 2). In htSNP rs242941A2 (SEQ ID NO:7) there was a trend for association between % decrease in HAM-A and the G allele versus the T allele (57.0±4.4% and 39.5±9.9%, respectively, P=0.07). The frequency of the G allele was 39/42 and the T allele was 13/42. In htSNP rs242941A1 (SEQ ID NO:7) there was G in the entire group of 80 subjects.

Allele frequencies of htSNP rs1876828 (SEQ ID NO:2), rs242939 (SEQ ID NO:6) and rs242941A2 (SEQ ID NO:7) were in Hardy Weinberg equilibrium, with no significant differences between actual and expected frequencies.

### Data De-Position and Data Sharing

All data reported here are deposited in PharmGKB, the Pharmacogenetics and Pharmacogenomics Knowledge Base. PharmGKB is a publicly available Internet research tool that is part of the nationwide collaborative research consortium, NIH Pharmacogenetics Research Network (PGRN). Its aim is to aid researchers in understanding how genetic variation among individuals contributes to differences in reactions to drugs.

A number of embodiments of the invention have been described. Nevertheless, it will be understood that various modifications may be made without departing from the spirit and scope of the invention. Accordingly, other embodiments are within the scope of the following claims.

1: A method for determining a subject's responsiveness to a psychiatric disorder therapy, wherein the subject is diagnosed with depression and anxiety, the method comprising the steps of:
(i) (a) providing a nucleic acid-comprising sample from the subject;
(b) analyzing the sample and detecting whether the subject is homozygous for a CRHR1, or “GAG,” haplotype (homozygous for the haplotype-tag single nucleotide polymorphisms (htSNPs) rs1876828 (SEQ ID NO:2), rs242939 (SEQ ID NO:6) and rs242941 (SEQ ID NO:7), wherein the presence of homozygosity for the CRHR1 genotype correlates with responsiveness to the therapy;
(ii) the method of (i), wherein the subject is diagnosed as having a psychiatric disorder comprising depression and anxiety;
(iii) the method of (ii), wherein the psychiatric disorder comprises major depression and anxiety disorder;
(iv) the method of any of (i) to (iii), wherein the psychiatric disorder therapy comprises treatment with an antidepressant agent;
(v) the method of (iv), wherein the antidepressant agent is selected from the group consisting of tricyclic antidepressants, selective serotonin reuptake inhibitors and CRHR1 antagonists;
(vi) the method of (i), wherein the nucleic acid-comprising sample comprises a blood sample, a saliva sample or a cell sample;
(vii) the method of (vi), wherein the cell sample comprises a cell from a biopsy, a buccal or a skin scraping or sample;
(viii) the method of (i), wherein the subject is selected from a subpopulation of patients;
(ix) the method of (viii), wherein the subpopulation of patients comprises a Mexican-American subpopulation;
(x) the method of (i), wherein the genotype of the subject with respect to the CRHR1, or “GAG,” haplotypes, is determined by amplification genotyping, in situ hybridization techniques, DNA array (biochip) analysis and/or direct DNA sequencing; or
(xi) the method of (x), wherein the amplification genotyping comprises polymerase chain reaction (PCR).

2. A method for screening a subject to determine the subject's responsiveness to a psychiatric disorder therapy comprising use of an agent which prevents or treats a psychiatric disorder associated with the corticotropin-releasing hormone receptor 1 (CRHR1) gene, comprising:
(i) (a) providing a nucleic acid-comprising sample from the subject;
(b) detecting whether the subject is homozygous for a CRHR1, or “GAG,” haplotype (homozygous for the haplotype-tag single nucleotide polymorphisms (htSNPs) rs1876828 (SEQ ID NO:2), rs242939 (SEQ ID NO:6) and rs242941 (SEQ ID NO:7), wherein the presence of homozygosity for the CRHR1 genotype correlates with responsiveness to the therapy;
(ii) the method of (i), wherein the subject is diagnosed as having a psychiatric disorder comprising depression and anxiety;
(iii) the method of (ii), wherein the psychiatric disorder comprises major depression and anxiety disorder;
(iv) the method of any of (i) to (iii), wherein the psychiatric disorder therapy comprises treatment with an antidepressant agent;
(v) the method of (iv), wherein the antidepressant agent is selected from the group consisting of tricyclic antidepressants, selective serotonin reuptake inhibitors and CRHR1 antagonists;
(vi) the method of (i), wherein the nucleic acid-comprising sample comprises a blood sample, a saliva sample or a cell sample;
(vii) the method of (vi), wherein the cell sample comprises a cell from a biopsy, a buccal or a skin scraping or sample;
(viii) the method of (i), wherein the subject is selected from a subpopulation of patients;
(ix) the method of (viii), wherein the subpopulation of patients comprises a Mexican-American subpopulation;
(x) the method of (i), wherein the genotype of the subject with respect to the CRHR1, or “GAG,” haplotypes, is determined by amplification genotyping, in situ hybridization techniques, DNA array (biochip) analysis and/or direct DNA sequencing; or
(xi) the method of (x), wherein the amplification genotyping comprises polymerase chain reaction (PCR).

3-12. (canceled)

13. A kit suitable for determining a subject's responsiveness to a psychiatric disorder therapy, wherein the subject is diagnosed with depression and anxiety, said kit comprising
(i) (a) material for determining whether the subject is homozygous for a CRHR1, or “GAG,” haplotype (homozygous for the haplotype-tag single nucleotide polymorphisms (htSNPs) rs1876828 (SEQ ID NO:2), rs242939 (SEQ ID NO:6) and rs242941 (SEQ ID NO:7); (b) suitable packaging material; and optionally (c) instructional material for use of said kit;
(ii) the kit of (i), wherein the material comprises at least one nucleic acid that specifically binds to the htSNPs rs1876828 (SEQ ID NO:2), rs242939 (SEQ ID NO:6) and rs242941 (SEQ ID NO:7);
(iii) the kit of (ii), wherein the nucleic acid that specifically binds to the htSNPs comprises at least one polymerase chain reaction (PCR) primer or a hybridization probe; or
(iv) the kit of (i) further comprising material to process a nucleic acid-comprising biological sample.

14-16. (canceled)

17. A method for determining a nucleotide polymorphism associated with a specific response to a psychiatric disorder therapy in an individual comprising
(a) sequencing all or a portion of the nucleotide sequence of chromosome 17q11-q22 from the individual, comparing the 17q11-q22 sequence from the individual to chromosome 17q11-q22 sequences of other individuals, and determining the presence of a nucleotide polymorphism in the sequenced 17q11-q22 sequences; and,
(b) correlating the specific response of the individual to a psychiatric disorder therapy with the presence or absence of the nucleotide polymorphism, wherein optionally the psychiatric disorder therapy is a drug therapy, or depression and anxiety.

18. A method for determining a nucleotide polymorphism associated with the presence of a psychiatric disorder in an individual comprising
(a) sequencing all or a portion of the nucleotide sequence of chromosome 17q11-q22 from the individual, comparing the 17q11-q22 sequence from the individual to chromosome 17q11-q22 sequences in individuals having the same psychiatric disorder, and determining the presence or absence of a nucleotide polymorphism in individuals having the same psychiatric disorder; and,
(b) correlating the diagnosis of the psychiatric disorder to the presence or absence of the nucleotide polymorphism, wherein optionally the psychiatric disorder therapy is a depression, or depression and anxiety.

19. A method for diagnosing the presence of a psychiatric disorder in an individual by determining a nucleotide polymorphism in an individual comprising
(a) sequencing all or a portion of the nucleotide sequence of chromosome 17q11-q22 from the individual, comparing the 17q11-q22 sequence from the individual to chromosome 17q11-q22 sequences in individuals having the same psychiatric disorder, and determining the presence or absence of a nucleotide polymorphism in individuals having the same psychiatric disorder; and,
(b) correlating the presence or absence of the nucleotide polymorphism to the presence or absence of a psychiatric disorder, wherein optionally the psychiatric disorder therapy is a depression, or depression and anxiety.

20. A method for determining a corticotropin-releasing hormone receptor 1 (CRHR1) protein or transcript isoform associated with a specific response to a psychiatric disorder therapy in an individual comprising
(a) determining the CRHR1 protein or transcript isoform or isoforms expressed in the individual, comparing the CRHR1 protein or transcript isoform or isoforms expressed in the individual to CRHR1 protein or transcript isoform or isoforms expressed in individuals having the same psychiatric disorder therapy; and,
(b) correlating the response of the individual to the psychiatric disorder therapy with the presence or absence of the expressed CRHR1 protein or transcript isoform or isoforms, wherein optionally the psychiatric disorder therapy is a drug therapy.

21. A method for determining a corticotropin-releasing hormone receptor 1 (CRHR1) protein or transcript isoform associated with the presence of a psychiatric disorder in an individual comprising
(a) determining the CRHR1 protein or transcript isoform or isoforms expressed the individual, comparing the CRHR1 protein or transcript isoform or isoforms expressed in the individual to CRHR1 protein or transcript isoform or isoforms expressed in individuals having the same psychiatric disorder, and determining the presence or absence of CRHR1 protein or transcript isoform or isoforms in individuals having the same psychiatric disorder; and,
(b) correlating the diagnosis of the psychiatric disorder to the presence or absence of the CRHR1 protein or transcript isoform or isoforms expressed in the individuals, wherein optionally the psychiatric disorder therapy is a depression.

22. A method for diagnosing the presence of a psychiatric disorder in an individual by determining what corticotropin-releasing hormone receptor 1 (CRHR1) protein or transcript isoforms are expressed in an individual comprising
(a) determining the CRHR1 protein or transcript isoform or isoforms expressed the individual, comparing the CRHR1 protein or transcript isoform or isoforms expressed in the individual to CRHR1 protein or transcript isoform or isoforms expressed in individuals having the same psychiatric disorder, and determining the presence or absence of CRHR1 protein or transcript isoform or isoforms expressed in individuals having the same psychiatric disorder; and,
(b) correlating the presence or absence of the CRHR1 protein or transcript isoform or isoforms expressed in the individuals to the presence or absence of a psychiatric disorder, wherein optionally the psychiatric disorder therapy is a depression.

23. A method for associating a protein isoform or its transcript isoform with a specific response to a psychiatric disorder therapy in an individual, wherein the protein is involved in the hypothalamic-pituitary-adrenal (HPA) axis pathway, comprising
(a) determining the protein or transcript isoform or isoforms expressed in the individual, comparing the protein or transcript isoform or isoforms expressed in the individual to protein or transcript isoform or isoforms expressed in individuals having the same psychiatric disorder therapy; and,
(b) correlating the response of the individual to the psychiatric disorder therapy with the presence or absence of the expressed protein or transcript isoform or isoforms,
wherein optionally the psychiatric disorder therapy is a drug therapy,
and optionally the protein is involved in the hypothalamic-pituitary-adrenal (HPA) axis pathway is corticotropin-releasing hormone (CRH), proopiomelanocortin (POMC), a protein involved in steroid synthesis or degradation, or a transcription factor involved in the HPA axis pathway.

24. A method for associating a protein isoform or its transcript isoform associated with the presence of a psychiatric disorder in an individual, wherein the protein is involved in the hypothalamic-pituitary-adrenal (HPA) axis pathway, comprising
(a) determining the protein or transcript isoform or isoforms expressed the individual, comparing the protein or transcript isoform or isoforms expressed in the individual to protein or transcript isoform or isoforms expressed in individuals having the same psychiatric disorder, and determining the presence or absence of protein or transcript isoform or isoforms in individuals having the same psychiatric disorder; and,
(b) correlating the diagnosis of the psychiatric disorder to the presence or absence of the protein or transcript isoform or isoforms expressed in the individuals,
wherein optionally the psychiatric disorder therapy is a depression
and optionally the protein is involved in the hypothalamic-pituitary-adrenal (HPA) axis pathway is corticotropin-releasing hormone (CRH), proopiomelanocortin (POMC), a protein involved in steroid synthesis or degradation, or a transcription factor involved in the HPA axis pathway.

25. A method for diagnosing the presence of a psychiatric disorder in an individual by determining what protein or transcript isoforms are expressed in an individual wherein the protein is involved in the hypothalamic-pituitary-adrenal (HPA) axis pathway, comprising
(a) determining the protein or transcript isoform or isoforms expressed the individual, comparing the protein or transcript isoform or isoforms expressed in the individual to protein or transcript isoform or isoforms expressed in individuals having the same psychiatric disorder, and determining the presence or absence of protein or transcript isoform or isoforms expressed in individuals having the same psychiatric disorder; and,
(b) correlating the presence or absence of the protein or transcript isoform or isoforms expressed in the individuals to the presence or absence of a psychiatric disorder, wherein optionally the psychiatric disorder therapy is a depression
and optionally the protein is involved in the hypothalamic-pituitary-adrenal (HPA) axis pathway is corticotropin-releasing hormone (CRH), proopiomelanocortin (POMC), a protein involved in steroid synthesis or degradation, or a transcription factor involved in the HPA axis pathway.


SEQUENCE LISTING (12 sequences)

SEQ ID NO: 1 - DNA, 51 bp, Artificial Sequence
  Primer
tcccctgctctgtagcctaaggacanttctcttggtccctcgcatggtgac

SEQ ID NO: 2 - DNA, 51 bp, Artificial Sequence
  Primer
gcagcatacccctagggacctaggancagggagggagagaggcagccctgg

SEQ ID NO: 3 - DNA, 51 bp, Artificial Sequence
  Primer
gtcccaacaggcctcacagccctganccccgctgcagggcccccgggtcct

SEQ ID NO: 4 - DNA, 51 bp, Artificial Sequence
  Primer
ccccaaccagagatgatgatgggggncaggggaggcaccaaaccctgggcc

SEQ ID NO: 5 - DNA, 51 bp, Artificial Sequence
  Primer
cctttcctgggatcacagagggaagngcgggggagcctagagagcaccaca

SEQ ID NO: 6 - DNA, 51 bp, Artificial Sequence
  Primer
gaacacggaggccacacaagagtggnttccaagtgaaggagtgaccaactc

SEQ ID NO: 7 - DNA, 51 bp, Artificial Sequence
  Primer
gggccaggaaccatgaaccagcgcgngtgggggcagcctcttcaggcctgg

SEQ ID NO: 8 - DNA, 51 bp, Artificial Sequence
  Primer
tgccatgaaggctccctgccctcaanccacatgtatcccctgcccagggct

SEQ ID NO: 9 - DNA, 51 bp, Artificial Sequence
  Primer
ggtggcccccacctctaggtagaggngtcctttctgtccacggttggcact

SEQ ID NO: 10 - DNA, 51 bp, Artificial Sequence
  Primer
gcagcatacccctagggacctaggagcagggagggagagaggcagccctgg

SEQ ID NO: 11 - DNA, 51 bp, Artificial Sequence
  Primer
gaacacggaggccacacaagagtggattccaagtgaaggagtgaccaactc

SEQ ID NO: 12 - DNA, 51 bp, Artificial Sequence
  Primer
gggccaggaaccatgaaccagcgcgggtgggggcagcctcttcaggcctgg